(19) United States
(12) Patent Application Publication
(10) Pub. No.: US 2009/0062647 A1
(43) Pub. Date: Mar. 5, 2009

(54) Method for extracting geometrical properties of a tubular cavity using low SNR echogram enhancement

(76) Inventors: Daniel H. Lange, Kfar Vradim (IL); Leonid Sternberg, Haifa (IL)

Correspondence Address: Pearl Cohen Zedek Latzer, LLP, 1500 Broadway, 12th Floor, New York, NY, 10036, US

(21) Appl. No.: 11/896,621

(22) Filed: Sep. 4, 2007

(51) Int. Cl. A61B 8/00

(52) U.S. Cl. 600/438

(57) ABSTRACT

A method for determining geometrical properties of a tubular cavity, the method comprising: transmitting a series of synchronized ultrasonic signals in predetermined timing from within the cavity; collecting echo data of the signals; analyzing the echo data to identify covariant components; extracting echo peaks from the identified covariant components; and calculating the geometrical properties of the cavity using the extracted echo peaks.

### FIELD OF THE INVENTION

The present invention relates to determining geometrical properties of a tubular cavity using a miniaturized probing device inserted into the cavity. More particularly, the present invention relates to extraction of low power boundary reflected ultrasonic echoes, for the purpose of determination of geometrical properties of the cavity, such as the inner and outer cavity dimensions.

### BACKGROUND OF THE INVENTION

Nature has identified ultrasonic waves as efficient means for distance estimation. Certain species like bats use ultrasonic wave transmission and reception as their primary space navigation system.

Man has adopted ultrasound as an appealing, non-invasive imaging modality. Ultrasonic echograms are used in a variety of applications dealing with measurement of geometrical dimensions of obscured objects, from imaging of fetus in the womb and to presentation of arterial blood flow. Other applications related to distance estimation modalities such as sonar are also in standard use.

Many fields require determination of information about the cavity of tubular structures, such as blood vessels. This information can include the thickness of the walls, the maximum and minimum internal diameters, and the location of a device inserted into the cavity relative to the cavity.

In anatomy, the cavities of tubular organs, such as veins and arteries, are called lumens. The present invention proposes a novel method for determining lumen properties by analyzing signals from a miniaturized probing device placed inside the lumen.

A typical prior art solution for analyzing lumen geometry and navigating within the lumen has been to use an ultrasonic navigation system. For example, in an ultrasound coronary investigation a full circle scan (2D) is used in order to estimate the lumen size of a blood vessel. Achieving the estimate requires a large amount of computational power and involves sophisticated image processing schemes.

In a disclosure incorporated herein as reference, PCT/IL02/00018 “Ultrasonic Transducer Probe”, Aharoni et al. (published as WO 03/057061) describe a compact cross-sectioned electromagnetic/acoustic arrangement for generating and detecting ultrasound waves using an electromagnetic waveguide. The acoustic generator comprises a source of electromagnetic radiation, a waveguide coupled to the source and at least one absorbing region defined in the waveguide, the region being selectively absorbing for portions of radiation meeting at least one certain criterion and having significantly different absorbing characteristics for radiation not meeting such criterion, the radiation being suitable for conveyance through the waveguide, where the absorbing region converts the radiation into an ultrasonic acoustic field. The phenomenon of converting electromagnetic radiation to ultrasound is comprehensively described in that disclosure.

In IL patent application no. 155329 (not yet published), there was disclosed a probing device for insertion into a duct having a physical structure to determine local parameters associated with the physical structure of the duct at a selected region of the duct, and in particular variations in the physical structure along a predetermined length of interest. The probing device comprises at least one of a plurality of waveguides incorporated in an elongated assembly designed to be inserted into the duct; at least one of a plurality of transmitters, spaced and distributed along a predetermined length of said at least one of a plurality of waveguides incorporated in the elongated assembly, each capable of independently transmitting an acoustic signal of predetermined characteristics; at least one of a plurality of receivers, spaced and distributed along a predetermined length of at least one of a plurality of waveguides incorporated in the elongated assembly, each capable of receiving echoes of the acoustic signal, reflected off the structure of the duct. When the transmitters generate an acoustic signal (each at a predetermined time), echoes of the signal are received by the plurality of receivers and received data associated with the echoes is processed by a processing unit to determine parameters of the physical structure at the region.

It has been shown (PCT/IL03/00584, published as WO 2004/008070) that an ultrasonic transceiver, inserted into a tubular cavity, may be used for assessment of geometrical properties of the cavity by calculation of time differences between the instant of transmission of an ultrasonic signal and the instant of reception of its associated reflected echoes.

In real-life situations, where the cavity might not necessarily be regular and smooth, reflectance echoes may collate and yield noisy, cluttered signals. Deciphering these collated echoes calls for an application-tailored signal-processing scheme to remove noise and clutter and thus enable identification of reflection timing differences from which the required geometrical dimensions may be calculated.

A main object of the present invention is to provide a method for analyzing signals from a miniaturized probing device inside a tubular cavity to determine properties of the cavity including the offset from the inner boundary of the probing device.

### SUMMARY OF THE INVENTION

There is thus provided, in accordance with a preferred embodiment of the present invention, a method for determining geometrical properties of a tubular cavity, the method comprising:

transmitting a series of synchronized ultrasonic signals from within the cavity, in a predetermined timing sequence,

collecting reflected echo data of the signals;

analyzing the echo data to identify covariant echo components;

extracting echo peaks from the identified covariant components; and

calculating cavity geometrical properties using the extracted echo peak timings.

Furthermore, in accordance with a preferred embodiment of the present invention, the echo peaks are primary peaks.

Furthermore, in accordance with a preferred embodiment of the present invention, the method further comprises extracting secondary echo peaks.

Furthermore, in accordance with a preferred embodiment of the present invention, the step of analyzing comprises:

high-pass filtering the echo data;

removing signal components below a predetermined threshold;

arranging the filtered and thresholded echo data into a data matrix;

decompressing the data matrix into three matrices employing Singular Value Decomposition (SVD) transformation;

extracting Eigenvectors and Eigenvalues from the SVD representation;

rectifying the Eigenvectors;

low-pass filtering the rectified eigenvectors;

detecting wave-front pattern;

identifying echo peaks in the wave-front pattern; and extracting the tubular dimensions using the extracted timing of said echo peaks.

Furthermore, in accordance with a preferred embodiment of the present invention, the high-pass filter is set to a value slightly below the frequency of the ultrasonic stimulus.

Furthermore, in accordance with a preferred embodiment of the present invention, the predetermined threshold is around 10% of a peak value.

Furthermore, in accordance with a preferred embodiment of the present invention, the low pass filter is set to be lower than half the ultrasonic stimulus frequency.

### BRIEF DESCRIPTION OF THE DRAWINGS

In order to better understand the present invention, and appreciate its practical application, the following Figures are provided and referenced hereafter. It should be noted that the Figures are given as examples only and in no way limit the scope of the invention. Like components are denoted by like reference numerals.

FIG. 1 illustrates a cylindrical cavity, showing its geometrical properties, with an ultrasonic transceiver within the cavity.

FIG. 2 illustrates a block diagram depicting the steps of a method for extracting low signal-to-noise ratio boundary reflected signals within the cavity, in accordance with the present invention.

FIG. 3 illustrates Gaussian distributions of the reflected signals around the theoretical reflectance angle within a cavity.

FIG. 4 illustrates an example of four low SNR echograms, used in the simulation for reconstruction of cylindrical cross-section internal and external diameters.

FIG. 5 presents an overlay of the extracted eigenvectors.

FIG. 6 illustrates the contribution of the extracted eigenvectors to data variance.

FIG. 7 illustrates the selected, compact time support eigenvector average, which is used for extraction of the echogram peaks.

FIG. 8 depicts the echogram peaks that are in turn used for calculation of the tubular cavity geometrical properties.

FIG. 9 illustrates the stationary echogram case (no jitter); the upper left plot is a simulated, noise-free echo signal, the lower left plot presents one echogram out of ten realizations, created by embedding the signal within white noise at an SNR of approximately 0 dB; the upper right plot shows the 110-echogram average, and the lower right plot presents the first multivariate eigenvector.

FIG. 10 illustrates the jittered echogram case; the upper left plot is a simulated, noise-free echo signal; the lower left plot presents one echogram out of ten realizations, created by embedding the jittered signal within white noise at an SNR of approximately 0 dB; the upper right plot shows the 10-echogram average, and the lower right plot presents the first multivariate eigenvector.

FIG. 11 illustrates a theoretical cavity, with an ultrasonic transmitting device and receiving device for transmitting and detecting echoes within the cavity. The transmitting device and receiving device may be encapsulated within a single transducer apparatus.

### DETAILED DESCRIPTION OF PREFERRED EMBODIMENTS

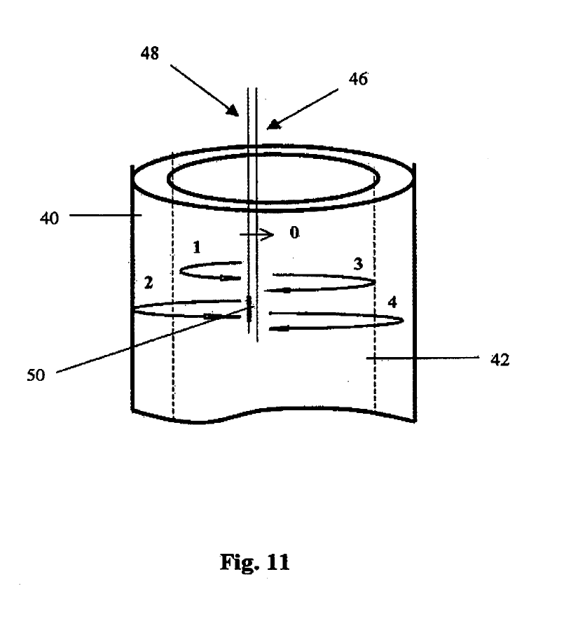

The method of the present invention is aimed at extraction of the geometrical properties of a tubular cavity, by means of analysis of synchronously repeating echograms, induced via a miniaturized ultrasonic transducer inserted into the cavity. Reference is made to FIG. 11 illustrating a cavity, with an ultrasonic transmitting device 48 and receiving device 46 for detecting echoes within the cavity. The position of the transducer 50 relative to the inner boundary of the cavity is also provided. By “tubular” it is meant, in the context of the present invention, any elongated cavity, defining a lumen (i.e. having circular or any closed amorphic cross section), possibly possessing structural irregularities. Such device is disclosed in PCT/IL02/00018 “Ultrasonic Transducer Probe”, Aharoni et al. (published as WO 03/057061), incorporated herein by reference.

The transducer transmits transient ultrasonic signals and receives echoes reflected from the tubular cavity boundaries (inner wall 42 and outer wall 40). These echoes depend on the exact location and orientation of the transducer within the cavity, however certain invariants relating to the inner and outer dimension of the cavity may be used for estimation of the cavity dimensions, as well as for estimation of the distance of the transducer from the inner boundary of the cavity. It is recommended that all echoes, pertaining to a single ultrasonic signal transmission, are collected prior to transmitting the consecutive ultrasonic signal.

A major obstacle relates to a low Signal to Noise Ratio (SNR) of the received ultrasonic signals, resulting from energy absorption by the cavity boundaries as well as from mechanical perturbations of the system. It is proposed to overcome the low SNR by using repeated, synchronized transmissions, followed by multivariate analysis of the echogram data, facilitating reduction of noise based on its random uncorrelated characteristic, and enhancement of the signal based on its multi-stationary character.

The ultrasonic echogram is comprised of three basic contributions: direct echoes bouncing off vessel boundaries, ultrasonic clutter, and noise; the echoes bouncing off vessel boundaries are the desired signal, the clutter represents superposition of multi-reflection echoes determined by lobe characteristics of the wide ultrasonic beam as well as by surface characteristic, and the noise encapsulates all other interferences. Thus, the echogram interpretability depends on elimination of the interfering noise and on the ability to distinguish direct boundary-reflected echoes from clutter.

We shall base the discussion on a perfect cylindrical cavity (see cross-section in FIG. 1).

An ultrasonic pulse wave is transmitted isotropically from point T. The first echo E₁, reflected from the inner boundary at point I₁, travels back towards the transceiver at point T. Some of the signal energy passes through the inner boundary at I₁ and is in turn reflected from the outer boundary at O₁; this reflection then travels back towards the transceiver at point T, creating a second echo signal E₂. In a similar manner, a third echo is reflected from the opposite inner boundary I₂, traveling back to the transceiver at point T. Some of the signal energy passes through the inner boundary at I₂ and is in turn reflected from the outer boundary at O₂; this reflection then travels back towards the transceiver at point T, creating a fourth echo signal E₄. We shall refer to these first four echoes as primary echoes. The primary echoes are used to calculate the inner and outer boundary diameters. The position of the transducer relative to the inner boundary of the cavity may also be calculated.

Determination and identification of the primary echoes are possible with the following limitations: (1) the second and third echoes may overlap or even exchange order of appearance, depending on the distance of the transducer from the boundaries as well as on the relative echo propagation velocities within the cavity and the wall; (2) the proposed multivariate analysis requires multi-stationary echoes, that is, echoes belonging to several stationary subgroups. In real-life situations the system might suffer from mechanical disturbances, like body motion related to heart-beat and breathing, which may result in echo variations.

This calls for usage of secondary echoes, being double reflection echoes traveling from the transducer, hitting the nearest boundary, bouncing backwards, passing through the transducer vicinity to hit the opposite boundary, and then bouncing back to be picked up by the transducer. Such secondary echoes are stationary with respect to transducer movement across transmissions, as they travel exactly twice the inner diameter irrespective of the exact location of the transducer within the cavity. In a similar manner, there exist double echoes traveling exactly twice the outer diameter, as well as double echoes traveling twice the sum of inner and outer diameter, irrespective of the exact location of the transducer within the cavity. Thus double reflections possess important invariant characteristic, which may be utilized in combination with the primary echoes to improve the primary echoes based analysis.

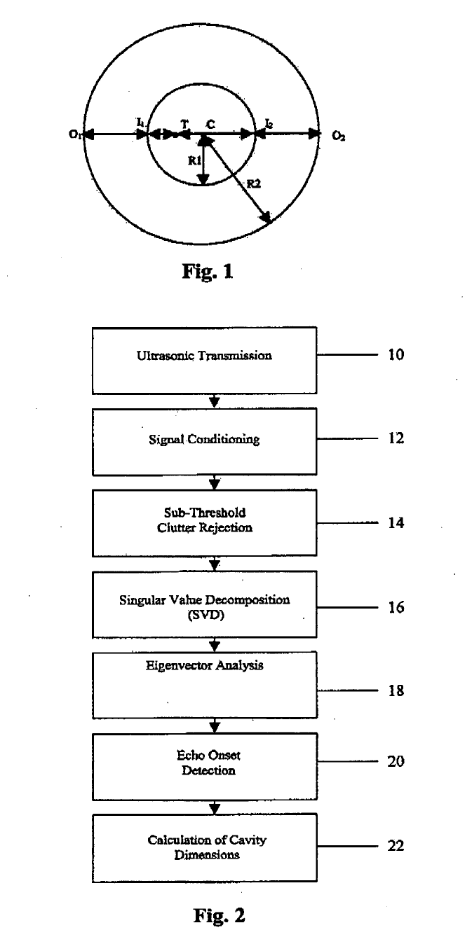

Internal and external cavity dimensions may be extracted from timing of the received echoes. However the echograms are masked in-part by noise and clutter interferences, limiting direct analysis of the raw data. Using repeated, synchronized ultrasonic pulses (signal conditioning 10, see FIG. 2), under multi-stationary mechanical conditions, yields correlated boundary reflected echoes, while noise contribution may be discriminated due to its stochastic nature. In addition, even the slightest mechanical perturbation may introduce substantial random effect on clutter characteristics, while its effect on the desirable boundary-reflected echoes is relatively small.

The present invention discloses a method for extraction of direct, boundary-reflected echoes from a batch of synchronous echograms, taken under multi-stationary mechanical conditions. Multi-stationarity is needed to ensure existence of one or more stationary echogram subgroups; this is required as the method is based on multivariate signal analysis, which basically consists of statistical techniques that consider several related random variables as a single entity and attempt to produce an overall result, taking the relationship among the variables into consideration. A block diagram of the proposed method is illustrated in FIG. 2.

The analysis is carried out on echogram reflection signals, resulting from synchronized ultrasonic radiation pulses 10—emanating from an ultrasonic transmitter and received by an ultrasonic receiver (for example such as described in WO 03/057061). The method of analysis presented herein consists of several stages, starting with signal conditioning 12, filtering and thresholding 14, followed by multivariate analysis 16,18, and concluded with wave-front detection 20 and calculation of the cavity dimensions 22. Following is a detailed description of the analysis steps.

Analysis Steps:

1) High-pass filtering of the raw consecutive echograms (12)

2) Elimination of below-threshold signal components (14)

3) Singular Value Decomposition (SVD) of synchronized echograms (16)

4) Extraction of Eigenvectors and Eigenvalues of SVD representation (18)

5) Full-wave rectification of the Eigenvectors (18)

6) Low-pass filtering of the rectified Eigenvectors (18)

7) Eigenvalue-weighted averaging of selected processed eigenvectors (18)

8) Wave-front echo onset detection (20)

9) Echo peak identification (22)

10) Calculation of cavity geometrical properties (22)

These steps are executed as follows:

The raw echograms are aligned synchronously and stored in a DATA matrix. Each column of the matrix, containing a single echogram, is high-pass filtered to reject baseline wandering. Preferably, the high-pass cutoff frequency is set to a value slightly lower than the frequency of the ultrasonic stimulus.

The filtered columns are thresholded to remove noise and clutter interference. Typically, values lower than 10% of peak value are rejected, but other threshold values may be acceptable too, depending on the level of noise and clutter.

The data matrix is decomposed into three matrices using an SVD (Singular Value Decomposition) transformation: DATA=U*S*V, where U and V are unitary matrices and S is a diagonal matrix. Eigenvectors and Eigenvalues are extracted from the SVD representation, as follows:

Eigenvalues: λᵢ=diag(S)

Eigenvectors: Wᵢ=Uᵢ*Sᵢ*Vᵢ

The Eigenvectors Wᵢ are rectified to ensure positive echo representation, and then low-pass filtered to smooth out the transition points and extract the envelope. The low-pass cutoff frequency is set according to the desired envelope temporal resolution, typically to a value lower than half the ultrasonic stimulus frequency.

The rectified eigenvectors may be ordered according to a compact time support criterion. One such possible criterion is echo duty-cycle. The ordered rectified eigenvectors are then selected and averaged to yield a representation of the ultrasonic echo pattern.

A first derivative of the ultrasonic echo pattern is taken, and then thresholded to yield a wave-front onset diagram.

The wave-front onset diagram is searched to identify the four primary reflection peaks, and the double reflection peaks.

The identified primary (and possibly also secondary) peak timings are utilized to calculate the geometrical properties of the cavity. The primary echo timings, for example, T₁-T₄, fulfill the following relations:

R₁=(T₁+T₃)/4Vᵢ;

R₂=(T₁+T₃)/4V₁+(T₂−T₁)/4Vₒ+(T₄−T₃)/4Vₒ.

Where Vᵢ and Vₒ represent the ultrasonic wave velocity in the inner and outer cavity, respectively.

To validate correct primary peak identification, the double-reflection secondary echoes may be used. For example, the first double echo, T_{d}, fulfills the following relation:

R₁=T_{d}/4Vᵢ (iii)

In a similar manner, additional double echoes may be used for further validation.

### Example Implementation (In MATLAB® Notation)

1) Let Xᵢ denote a single echogram. The high-passed echogram Yᵢ is achieved by: Yᵢ=filtfilt(hp,1,Xᵢ), where hp are the high-pass filter coefficients.

2) Thresholding:

Zᵢ=Yᵢ>(Yᵢ>threshold);

3) Calculation of SVD transform:

[USV]=svd(echo_matrix);

where echo_matrix columns are comprised of the processed echograms Zᵢ.

4) Eigenvectors are extracted, rectified, and low-pass filtered as follows:

EIGVEC(:,i)=ABS(V(:,i)*U(i,:)*S(:,i|);

EIGVEC_{—}LP(:,i)=filtfilt(lp,1,EIGVEC(:,i));

5) Weighted averaging:

EIGVAL=diag(S);

WEIGHTED_AVG=mean(EIGVAL.*VEC);

6) Echo onset detection:

WAVE_FRONT=WEIGHTED_AVG(2:N)−WEIGHTED_AVG(1:N−1);
ECHO_ONSET=WAVE_FRONT.*(WAVE_FRONT>0);
ECHO=ECHO_ONSET.*(ECHO_ONSET>THRESHOLD)

7) Peak search:

```
i=2;
while i<length(WAVE_FRONT),
    if WAVE_FRONT(i)>WAVE_FRONT(i−1) &
    WAVE_FRONT(i)>WAVE_FRONT(i+1),
        k=k+1;
        peak(k)=i;
        end;
    end;
```

8) Extraction of geometrical properties:

R₁=(T₁+T₃)/4Vᵢ;

R₂=(T₁+T₃)/4Vᵢ+(T₂−T₁)/4Vₒ+(T₄−T₃)/4Vₒ

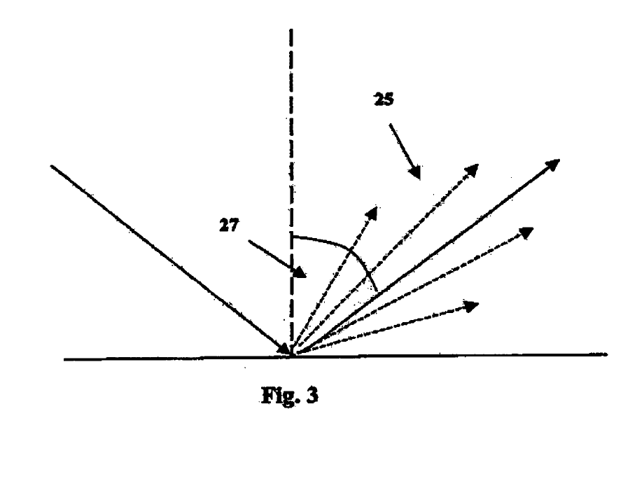

The model considered in the following simulation makes use of ultrasound velocity, reflection, and absorption coefficients in the participating media, and assumes Gaussian distributions of the reflected signals 25 around the theoretical reflectance angle 27 (FIG. 3). In addition, the model emulates low SNR conditions by masking the simulated ultrasonic echo signals with additive white random noise.

The simulation includes emulation of ultrasonic pulses, transmitted isotropically in a cylindrical, double-boundary cavity, imitating conditions expected to be encountered in a cylindrical blood vessel.

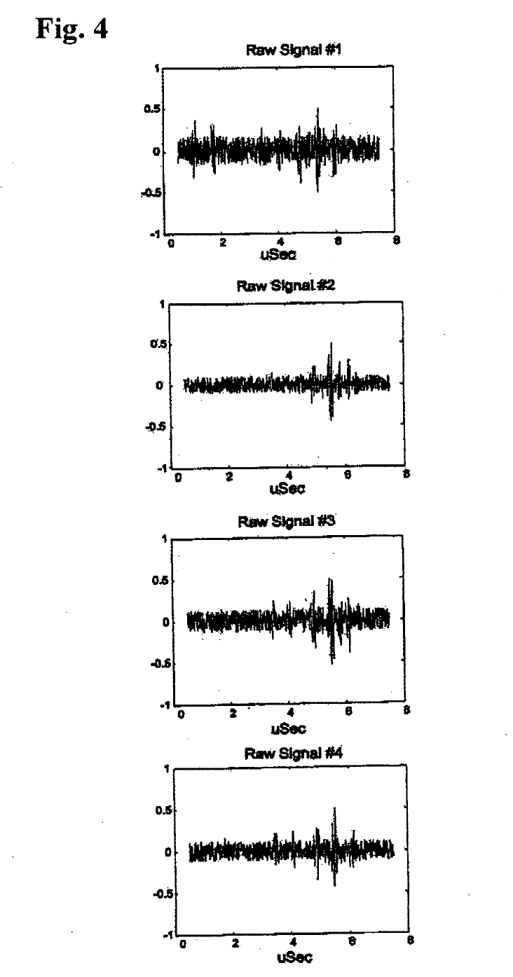

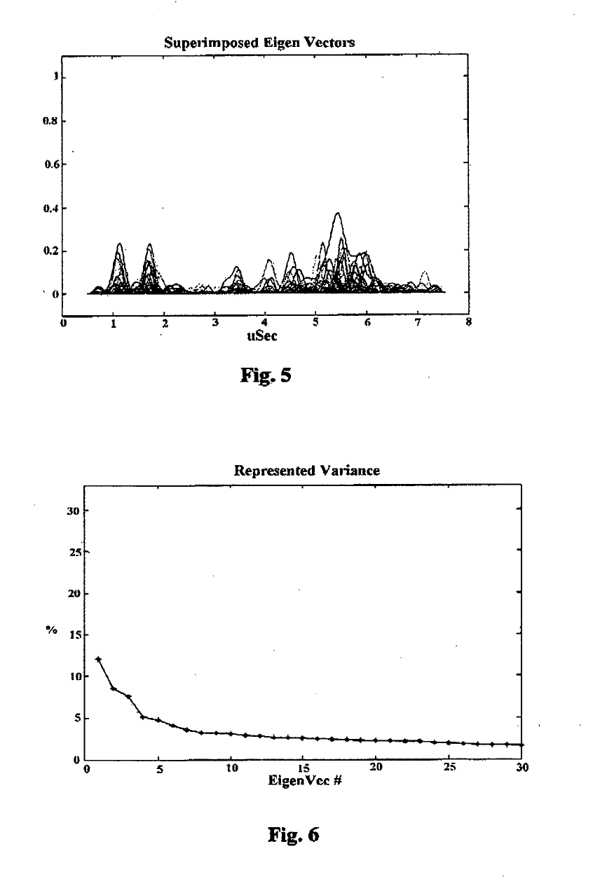

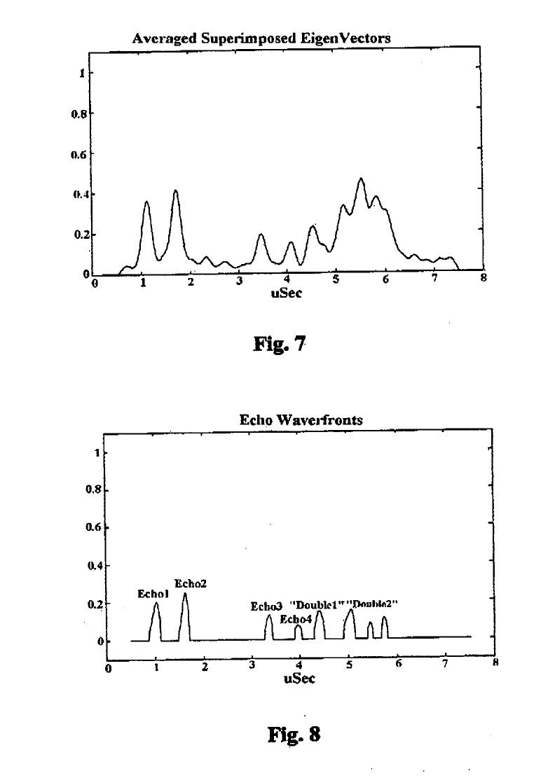

FIG. 4 presents an example of a low SNR echogram, used in the simulation for reconstruction of cylindrical cross-section internal and external diameters. FIG. 5 presents an overlay of the extracted eigenvectors, the contribution of which to data variance is shown in FIG. 6. FIG. 7 presents the selected, compact time support eigenvector average, which is used for extraction of the echogram peaks, as depicted in FIG. 8. These peaks are in turn used for calculation of the tubular cavity dimensions. Note the primary echo peaks denoted by: Echo1, Echo2, Echo3, Echo4, and the secondary “double” peaks denoted by: “Double1”, “Double 2”.

Multivariate analysis offers significant advantages over conventional averaging. While averaging is an effective tool for enhancing repeating, deterministic signals embedded in noise, variable signals are distorted by averaging. Multivariate analysis is a powerful tool for enhancing variable signals, provided that the signals may be sub-grouped to clusters and co-vary within each cluster.

The following description compares the performance of multivariate analysis and averaging, using simulated echogram signals. The simulation presents the advantage of multivariate analysis when the signals suffer from latency jitter, as expected in real-life situations due to sensor motion during the measurement. With jitter increase, averaging degrades rapidly while the multivariate representation continues to capture the main echo characteristics.

The signal is constructed using simulated echogram patterns. The echogram patterns are embedded within white noise at an SNR of approximately 0 dB (1:1). Echo timing variations are implemented by using random time shifts, ranging up to 25 sample points, equivalent to 0.25 uSec at a sampling frequency of 100 MHz. The ultrasonic pulse, taken from a physical ultrasonic transceiver system, lies between 15 MHz and 20 MHz. Ten repetitions are used for the averaging and multivariate analysis. These parameters are given as an example only and in no way limit the scope of the present invention.

The Singular Value Decomposition (SVD) transform is used to extract the eigenvectors of the signal covariance matrix. The eigenvectors are ordered in a descending order according to the amount of signal variance they represent. To demonstrate the advantage of multivariate analysis over conventional averaging, analysis of the first eigenvector, which represents most of the signal variance, is presented. In cases of several signal clusters, subsequent eigenvectors should also be used.

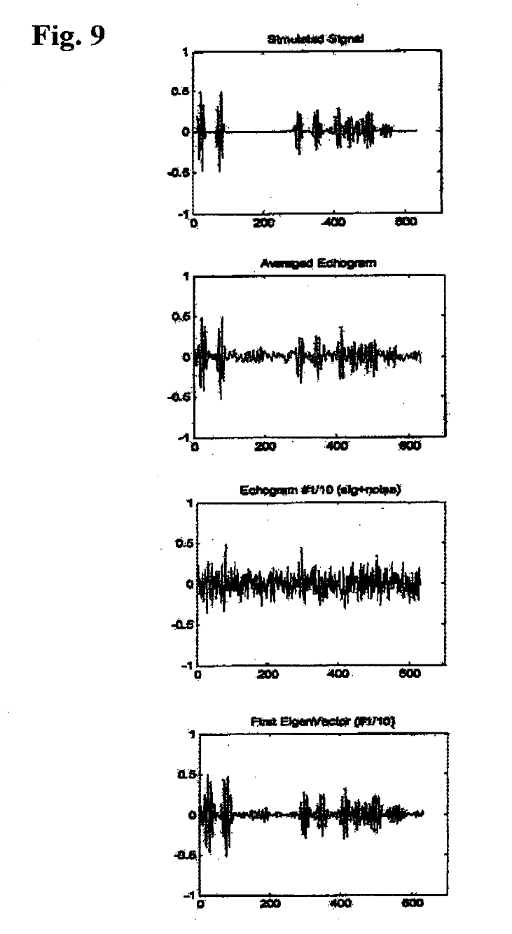

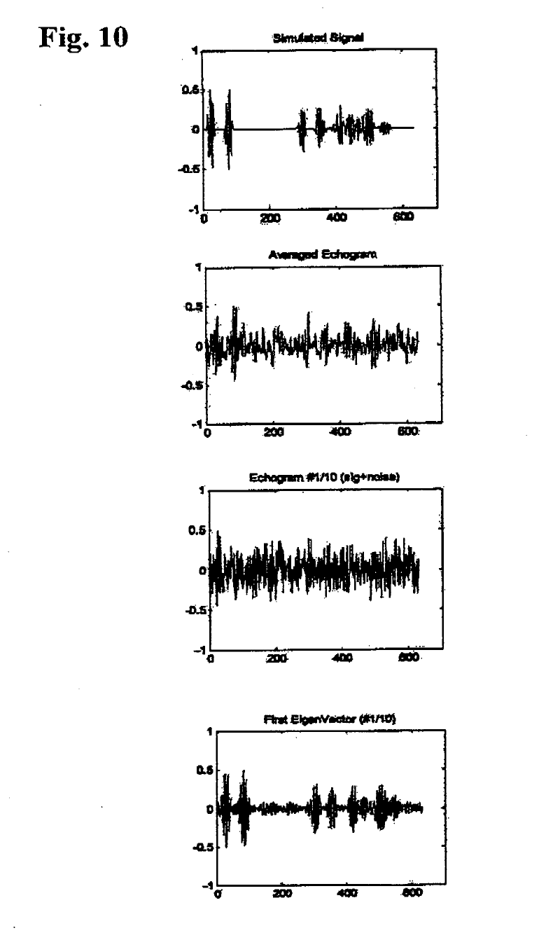

FIGS. 9 and 10 are divided into four plots. The upper left plot is a simulated, noise-free echo signal. The lower left plot presents one echogram out of ten realizations, created by embedding the stationary or jittered signal within white noise, at an SNR of approximately 0 dB. The upper right plot shows the 10-echogram average, and the lower right plot presents the first multivariate eigenvector.

With stationary echograms (no jitter), the average waveform and the eigenvector appear similar (FIG. 9). With jittered echograms, the average waveform becomes significantly distorted, while the eigenvector representation maintains an adequate representation of the embedded, jittered echogram (FIG. 10).

It is understood that there may be cases where some of the peaks (either primary or secondary peaks) will be overlapping or masked by background noise, resulting in the retrieval of only some of the anticipated peaks. As a result, the obtained cavity characteristic be partial, nevertheless in most cases there will be sufficient information to allow extraction of the major cavity characteristic.

The method of the present invention, although not limited to this application only, may strongly appeal to the investigation of blood vessels or other body tubular cavities.

The above mathmatical description was based on the assumption that the cross-section of the cavity is circular. Where the cavity is non-circular, the method of the present invention will in fact determine the dimensions of the largest circle that may be engulfed within the cavity at the location of measurement. This is important information to allow, for example, a surgeon to determine the minimal aperture that is available for blood to flow through, or to determine the largest diameter of a surgical tool which may be inserted through the cavity at that location. The method of the present invention may be used, for example, to obtain the geometrical properties of blood vessels, urinary tract, reproduction tract, intestinal, respiratory pathway, and other such bodily cavities.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the scope of the present invention.

What is claimed is:

1. A method for determining geometrical properties of a tubular cavity, the method comprising:
transmitting a series of synchronized ultrasonic signals in predetermined timing from within the cavity,
collecting echo data of the signals;
analyzing the echo data to identify covariant components;
extracting echo peaks from the identified covariant components; and
calculating the geometrical properties of the cavity using the extracted echo peaks.

2. The method of claim 1, wherein the step of analyzing comprises:
high-pass filtering the echo data;
removing signal components below a predetermined threshold;
arranging the filtered and thresholded echo data into a data matrix;
decompressing the data matrix into three matrices employing Singular Value Decomposition (SVD) transformation;
extracting Eigenvectors and Eigenvalues from the SVD representation;
rectifying the Eigenvectors;
low-pass filtering the rectified Eigenvectors;
detecting wave-front echo onset;
identifying echo peaks, and
calculating the cavity geometrical properties.

3. The method of claim 1, wherein the echo peaks are primary peaks.

4. The method of claim 1, further comprising extracting secondary echo peaks.

5. The method of claim 2, wherein the high-pass filter is set to a value slightly below the frequency of the ultrasonic stimulus.

6. The method of claim 2, wherein the predetermined threshold is around 10% of peak value.

7. The method of claim 2, wherein the low pass filter is set to be lower than half the ultrasonic stimulus frequency.

8. The method of claim 1, used to determine geometrical properties of cavity in a living body.

9. The method of claim 8, used to determine geometrical properties of a blood vessel.

10. The method of claim 8, used to determine geometrical properties of a urinary tract.

11. The method of claim 8, used to determine geometrical properties of a respiratory pathway.

12. The method of claim 8, used to determine geometrical properties of an intestinal tract.

13. The method of claim 8, used to determine geometrical properties of a reproduction tract.
